(19) United States
(12) Patent Application Publication
(10) Pub. No.: US 2012/0322718 A1
(43) Pub. Date: Dec. 20, 2012

(54) Amygels in organic solvent for biomedical applications

(76) Inventor: Elizabeth Barker, Southaven, MS (US)

(21) Appl. No.: 13/507,264

(22) Filed: Jun. 18, 2012

Related U.S. Application Data
(60) Provisional application No. 61/520,933, filed on Jun. 17, 2011.

(51) Int. Cl. A61K 47/36; A61K 38/02; A61P 35/00; A61K 31/7088; A61K 31/194; A61K 47/48; A61K 31/715

(52) U.S. Cl. 514/1.1; 514/778; 514/54; 514/44 R; 514/574

(57) ABSTRACT

A starch-based Amygel for the controlled delivery of a biologically active agent is described. The Amygel comprises a dispersed phase, and a dispersion medium consisting substantially of an organic solvent. The dispersed phase includes a polymeric network comprising a hydrolyzable polymeric substance derived from starch, and a cross-linker. In various embodiments, the polymeric starch-derived network includes amylose, amylopectin, soluble starch or a combination thereof. The cross-linker is a molecule having at least two carboxyl moieties. The hyrogel is biocompatible and biodegradable, and suitable for loading with biologically active agents including small molecule therapeutics, macromolecules such as proteins, polysaccharides and nucleic acids. Upon administration to a host animal, the Amygel biodegrades, releasing as degradation products only naturally-occurring sugar molecules that are non-toxic and non-immunogenic to the host.

### CLAIM OF PRIORITY

The applicant claims for this application the priority date established by provisional patent applications 61/520,933, filed on Jun. 17, 2011.

### FIELD OF THE INVENTION

The invention relates to the use of biodegradable carbohydrate-based Amygels as delivery systems for delivery of therapeutic agents for biomedical applications.

The invention relates to the field of delivery systems for biologically active agents. More specifically, the invention relates to the use of biodegradable carbohydrate-based Amygels useful for the delivery of biologically active agents including small molecules, macromolecular biologics such as therapeutic proteins, and whole cells.

### BACKGROUND OF THE INVENTION

Recent advances in medicine have gone beyond oral and injectable systemic delivery of therapeutic pharmaceutical agents to treat disease, and there is ongoing interest in pursuing avenues of more directed drug delivery to the site of affected organs, tissues, and cells. At the same time, it is recognized that there is an advantage in providing drug delivery vehicles capable of sustained release of the drug or biologically active agent. The fields of polymer engineering and biomaterials research have contributed greatly to advances in this area of medicine and pharmaceutical science by providing novel materials and material systems that can be used as biocompatible and/or biodegradable vehicles for drug delivery (Ratner 2004).

It is recognized that a therapeutic agent can interact with the body at one of three sites: within the circulation or an interstitial space; at membrane receptors on the surface of the cell; or at various sites of cellular machinery within the cell. A wide variety of polymeric biomaterials is available and routinely used for delivering drugs to these sites. A major advantage is provided by drug delivery systems that can release active therapeutic agents in a controlled manner. When drugs are taken orally, the blood plasma concentration rapidly rises, and then exponentially decays as the drug is metabolized and eliminated from the body. For a given drug of interest, there is a known concentration above which the drug could be toxic or have adverse effects, and conversely there is a concentration below which the drug is not likely to be therapeutically effective. The difference between these two concentration limits is known as the therapeutic index. Increasing the size of the dose can serve to increase the time during which the concentration of the drug is above the minimum effective concentration; however this can also increase the chances of approaching toxic blood plasma concentrations and side effects. Desired drug concentration can also be maintained by periodic doses, but this is inconvenient and usually is impeded by poor patient compliance. For these reasons, there has been great interest in developing controlled-release formulations and devices that can maintain a desired blood plasma level for long periods of time, without reaching a toxic level or dropping below the minimum effective level (Ratner 2004).

Hydrocarbon based gels have been used as drug delivery systems, including systems designed for drug delivery to a localized target in the body. “Gelation” is a term that refers to the cross-linking of many polymer chains within a hydrocarbon gel, into progressively larger branched polymers extending ultimately to a single continuous network. Hydrocarbon gels can be formed by either covalent bonds produced by the simple reaction of one or more co-monomers, by physical cross-links from polymer chain entanglements, by association bonds such as hydrogen bonds or strong van der Waals interactions between chains, or by crystallites bringing together two or more chains irrespective of their method of synthesis, all hydrocarbon gels are three-dimensional networks that are able to swell in solvent (Ratner 2004).

In polymer science it is recognized that the chemical structure of a polymer system determines the mechanical properties of the material. Specific chemical parameters such as the identity of the monomers used, the polymerization conditions, the cross-link density, the degree of swelling, and the type of medium in which the material is swollen are all factors that contribute to achieving the ultimate mechanical properties of the polymeric product. For biodegradable biomaterial systems, one very important aspect of material design is biocompatibility, including both that of the intact material, and that of the degradation products of the system. Most hydrocarbon gels presently available for biomedical applications contain at least some synthetic polymers within them. Unfortunately, synthetic polymers are known to produce toxic breakdown products when they are degraded.

Naturally occurring polysaccharides are one of the most abundant and diverse families of biopolymers on Earth. Recently, chemically modified polysaccharides have been considered for their suitability in controlled-release drug carrier systems. The repeating unit of polysaccharides may contain hydroxyl, carboxyl, amino, or sulfate functional groups, providing sites for the chemical modification of the polysaccharides.

Starch is a naturally occurring polysaccharide comprising long-chain polymers of D-glucose. Certain starch components have been contemplated for use in a biodegradable drug delivery system because of several natural advantages provided by starch, including biocompatibility, biodegradability, non-toxicity, and ready availability from abundant sources (Dumitriu 1996). However, most hydrocarbon gels in present use, or theoretically contemplated for use as biodegradable drug delivery devices, are lacking in one or more desirable properties, including construction from exclusively natural polymeric materials which are degradable into only natural products that are recognized by the body.

Further, hydrocarbon gels in present use, or theoretically contemplated for use as biodegradable drug delivery devices, do not allow for delivery of therapeutic agents that are hydrophobic.

### SUMMARY OF THE INVENTION

The invention provides a novel biodegradable starch-based amygel in organic solution for delivery of hydrophobic biomedical therapeutic agents. The inventive amygel is designed for controlled-release delivery of hydrophobic drugs or biologically active agents contained within the amygel. The invention incorporates several advantageous features. First, the amygel can be engineered to accommodate different types of therapeutic molecules including small molecules, macromolecules such as proteins, and whole cells. Importantly, the degradation characteristics of the amygel, and hence its drug release kinetics, can be customized to meet the specifications of a particular application. This capability greatly expands the realm of its therapeutic uses. Envisioned uses of the amygel drug delivery system include inter alia wound healing, hormone therapy, and cancer treatment, but the invention is not so limited.

One preferred embodiment of a starch-based amygel in accordance with the present invention is a controlled release drug delivery system that provides the advantage of adjustable release kinetics, which can be specified during synthesis of the amygel by varying the concentrations of the polymeric components of the starting materials. More particularly, the inventive amygel is a starch-based biodegradable amygel comprising an interpenetrating network of physically entangled starch polymer chains cross-linked with a chemical cross-linker. During synthesis of the gel, the chemically reactive carboxyl groups of the chemical cross-linker react with the —OH groups of the starch chains via condensation, creating ester linkages within the system.

Upon interaction with a biological environment, the ester linkages in the amygel degrade according to the same hydrolytic mechanism of the main chain backbone, resulting in the release of the chemical cross-linker while the hydrolysis of the acetal bonds of the starch results in the generation of glucose monomers, maltose dimers, and maltotriose trimers. By virtue of this design, no synthetic products are released into the body as a result of the degradation of the hydogel. Rather, all of the breakdown products are sugars that can be safely consumed by the surrounding cells in the tissue. Studies described herein demonstrate that with the addition of the cross-linker, the viscosity of a starch-based amygel increases. With increased cross-link concentration, the degradation time of the system is extended. As a further advantage, the inventive amygels can be loaded with different classes and sizes of drugs, which are released from the gels over time, under physiological conditions.

Accordingly, and in one aspect, the invention provides an amygel for the controlled delivery of a biologically active agent. The amygel comprises a dispersed phase and a dispersion medium. The dispersed phase includes a polymeric network comprising a polymeric substance derived from starch, and a cross-linker. In various preferred embodiments, the polymeric starch-derived substance comprises amylose, amylopectin, or a combination thereof. In other embodiments, the starch-derived substance is soluble starch. The dispersion medium consists substantially of organic solvent. In various preferred embodiments, the organic solvent is dimethyl-sulfoxide (DMSO).

In some preferred embodiments of the amygels, the cross-linker is covalently bound to the starch-derived polymeric substance by ester linkages. Suitable cross-linkers can be molecules having at least two carboxyl moieties, including dicarboxylic acids, tricarboxylic acids, and alpha hydroxy acids.

Some embodiments of the amygels include a biologically active agent, which can include a small molecule, a macromolecule such as a protein, polysaccharide, or nucleic acid, whole cells, or other biologically active agents. Some embodiments of the amygels include more than one type of biologically active agents.

Certain preferred embodiments of the amygels include a cross-link that has the potential to cause a biological effect following the controlled release in vivo of the crosslink from the amygel.

The inventive starch-based amygels are biodegradable. Upon administration to a host animal, and following biodegradation of the amygel, the breakdown products that are released from the amygels are exclusively naturally-occurring molecules and as such are non-toxic and non-immunogenic to the host animal.

The inventive starch-based amygels are produced in organic solvents, such as DMSO. The inventive amygels are insoluble in organic solvents and swell while absorbing organic solvents when placed in contact with organic solvents. One advantage of the inventive amygel is that hydrophobic biological agents are incorporated into the gel by dissolving such agents into organic solvents such as DMSO, followed by absorption of said organic solvent.

These and other advantages of the invention are further described in the specification.

### DETAILED DESCRIPTION

The present invention provides in one aspect an Amygel for biomedical applications, which is described more fully hereinafter. This invention may be embodied in many different forms and should not be construed as limited to the specific embodiments described herein.

### DEFINITIONS

An “Amygel,” as the term is used herein, refers to a gel that is composed usually of one or more polymers. An Amygel is a colloidal system in which the “dispersed phase” (colloid) has combined with the “dispersion medium” (organic solvent) to produce a viscous jelly-like product. An Amygel refers to a polymeric system essentially forming one continuous network. The polymeric network has the ability to absorb solvent without damage to the network. Amygels in accordance with the present invention are biodegradable by hydrolysis, i.e., the polymeric network is “hydrolyzable.” The “hydrolyzable polymeric substance” comprising the dispersed phase network is derived from starch, or starch derivatives. Starch can be hydrolyzed to simple sugars by enzymatic reactions, including those that occur naturally during biodegradation processes in vivo in animals, including humans.

“Analogue,” as the term is used herein refers to a chemical compound with a structure similar to that of another but differing from it in respect to a certain component; it may have a similar or opposite action metabolically.

“Biodegradable,” as the term is used herein refers, to the ability of a material to be broken down in a physiological environment.

“Biopolymer,” as the term is used herein, refers to a polymeric substance (such as a protein or polysaccharide) produced by a living organism or formed in a biological system. A biopolymer is a macromolecule. i.e., a long chain molecule made up of many individual repeat units. The individual repeat units or monomers which make up the polymer chain can consist of sugars, amino acids, or nucleotides. Biopolymers can be found in plants and animals and include carbohydrates, proteins, and DNA. Preferred biopolymers of use in the invention are polysaccharides such as starch, and its derivatives. Starch is produced by all green plants. Common sources of starch include corn, maize, casava, and potato.

“Complex” is a molecular entity formed by loose association involving two or more component molecular entities (ionic or uncharged), or the corresponding chemical species. The bonding between the components is normally weaker than a covalent bond.

“Derive” means to obtain (a chemical substance) actually or theoretically from a parent substance.

“Parenteral administration” refers to administration to an animal, including a human subject by any route other than the alimentary canal, including, e.g., intravenous, subcutaneous intramuscular, topical, intraocular, and nasal administration.

“Polyol” refers to an alcohol containing more than two hydroxyl groups (e.g., sugar alcohols, inositol); also known as polyhydric alcohol.

“Polysaccharide” is a carbohydrate that can be decomposed by hydrolysis into two or more molecules of monosaccharides; especially: one containing many monosaccharide units and marked by complexity (such as cellulose, starch, or glycogen).

“Solvation” is any stabilizing interaction of a solute (or solute moiety) and the solvent or a similar interaction of solvent with groups of an insoluble substance. Such interactions generally involve electrostatic forces and van der Waals forces, as well as chemically more specific effects such as hydrogen bond formation.

“Starch” is any one of a group of carbohydrates or polysaccharides, of the general composition (C₆H₁₀O₅)ₙ occurring as organized or structural granules of varying size and markings in many plant cells. Starch hydrolyzes to several forms of dextrin and glucose. Its chemical structure is not completely known, but the granules consist of concentric shells containing at least two fractions: an inner portion called amylose, and an outer portion called amylopectin.

“Soluble starch” refers to starch molecules that have been degraded. Soluble starch is derived from naturally occurring starch, for example, by acid hydrolysis with hydrochloric acid, resulting in a product having a molecular weight of about 300 g/mole. Amylopectin and soluble starch are both long chain molecules composed of glucose monomers; however they differ greatly in size. In contrast to soluble starch, the molecular weight of amylopectin can be as high as 10⁸ g/mole.

Other definitions are presented as necessary in the description that follows. The present invention provides in one aspect an Amygel for use in biomedical applications such as drug delivery. In certain embodiments of the present invention, the colloid includes a polymeric network having both hydrophilic surfaces and hydrophobic surfaces. These surfaces enable the polymeric network to retain solvent via intermolecular forces (e.g., hydrogen bonding). The polymeric network has the ability to interact with organic solvents without being dissolved by such solvents.

Biodegradable Amygels Comprising Natural Polymers

Amygels prepared in accordance with the present invention comprise polymers that are derived exclusively from natural sources (also referred to hereinafter as “natural polymers”). As the term is used herein, a “natural polymer” is meant to include a macromolecule that occurs in polymeric form in nature. Natural polymers included in the Amygels of the invention are isolated polymers that are derived exclusively from starting materials that are natural polymers. For example, a particularly preferred natural polymer useful in the invention is starch, which is a mixture of complex carbohydrates that is stored in abundance in e.g., seeds of plants such as corn. Starch that is isolated from natural sources is comprised of two main polysaccharides that form natural polymers with a helical shape, i.e., amylose and amylopectin.

Amygels in accordance with the present invention are biodegradable. As a biodegradable drug delivery vehicle in a biological environment, natural polymers, as compared with synthetic polymers, or polymers made with a combination of natural and synthetic macromolecules, offer several advantages. Being very similar, and in some cases identical, to endogenous macromolecular substances, the molecules of the biological carrier can be recognized and metabolically processed by the biological environment. Thus, by incorporating only natural polymers, the amygels of the invention are designed to be degradable by the body into naturally-occurring metabolic products, using the body's own mechanisms for breaking down these natural polymers.

This use of natural polymers provides an important advantage over drug delivery vehicles in the prior art that include synthetic polymers to some extent. Many problems are known to be associated with the use of synthetic polymers for this purpose, including toxicity of breakdown products, stimulation of a chronic inflammatory reaction upon implantation and breakdown of the product, and lack of recognition by cells. Each of these problems is suppressed or eliminated in the Amygels of the invention, which incorporate only natural polymers.

As a further advantage, the similarity of the natural polymers or their derivatives to naturally occurring substances introduces the interesting capability of designing biomaterials that function biologically at the molecular level, rather than at the macroscopic level.

A particularly advantageous characteristic of natural polymers is their ability to be degraded by naturally-occurring enzymes, ensuring that the implant made of a natural polymer eventually will be metabolized by physiological mechanisms. Although this property may at first appear as a disadvantage since it detracts from the durability of the implant, in fact it can be used advantageously for applications in the biomaterials field in which it is desirable to deliver a specific drug on a temporary basis, following which the implant degrades completely and is disposed of by largely normal metabolic processes.

Amygels for biomedical applications in accordance with the present invention incorporate a polysaccharide, which is a class of biomaterials that are naturally occurring polymers. The term “polysaccharide” refers to compounds made up of many hundreds or even thousands of monosaccharide units per molecule. As in disaccharides, these units are held together by glycoside linkages, which can be broken by hydrolysis. Polysaccharides are naturally occurring polymers, which can be considered as derived from aldoses or ketoses, by polymerization with loss of water. A polysaccharide has the general formula (C₆H₁₂O₅)ₙ.

By far the most abundant polysaccharides are cellulose and starch. Both are produced in plants from carbon dioxide and water by the process of photosynthesis, and both are made up of D-(+)-glucose units. Cellulose is the chief structural materials of plants, giving plants rigidity and form. Cellulose is likely the most widespread organic material on Earth.

Particularly preferred polysaccharides are starch, or starch derivatives including amylose and amylopectin. Starch makes up the reserve food supply of plants, and occurs chiefly in seeds. It is more water-soluble than cellulose, more easily hydrolyzed, and hence more readily digested (Morrison and Boyd 1983). Starch occurs as granules whose size and shape are characteristic of the plant from which the starch is obtained. When intact, starch granules are insoluble in cold water. If the outer membrane has been broken, for example by grinding, the granules swell in cold water and form a gel. When the intact granule is treated with warm water, a soluble portion of the starch diffuses through the granule wall. In hot water, starch granules swell to such an extent that they can burst.

Polysaccharide mixtures occur naturally, and it has been recognized that binary carbohydrate gels can be used as models for complex cellular structures involving the recognition step in certain host-pathogen interactions (see, for example, Dumitriu 1996).

In general, the term “starch” refers to a composite natural product comprising about 20% of a water-soluble fraction called amylose, and about 80% of a water-insoluble fraction called amylopectin. These two fractions appear to correspond to different carbohydrates of high molecular weight and formula (c6H12O5)n. Upon treatment with acid or under the influence of enzymes, the components of starch are hydrolyzed progressively to dextrin, a mixture of low molecular weight polysaccharides, (+) maltose, and finally D-(+)-glucose. Both amylose and amylopectin are made up of D-(+)-glucose units, but differ in molecular size and shape.

The only disaccharide that is obtained by the hydrolysis of amylose is (+)-maltose; the only monosaccharide that is obtained is D-(+)-glucose. To account for this, it has been proposed that amylose is made up of chains of many D-(+)-glucose units, each unit being joined by an alpha-glycosidic linkage to C-4 of the next one (Morrison and Boyd 1983). Amylose, shown in Formula 1 below, is made up of long chains each containing 1000 or more D-glucose units joined together by alpha linkages. There is little or no branching of the chain.

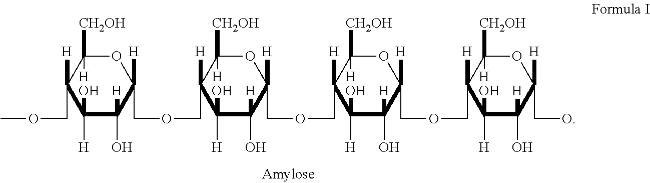

Amylopectin, like amylose, is made up of chains of D-glucose units, with each unit being joined by an alpha-glycoside linkage to C-4 of the next D-glucose unit. However, the structure of amylopectin is more complex than that of amylose. Molecular weight determination of amylopectin using physical methods as described, shows that there are up to a million D-glucose units per molecule of amylopectin. This molecule has a highly branched structure, consisting of several hundred short chains of about 20-25 D-glucose units each. One end of each of these chains is joined through a C-1 to a C-6 on the next chain (Morrison and Boyd 1983). A portion of the chemical structure of amylopectin, showing a branch point, is depicted in Formula:

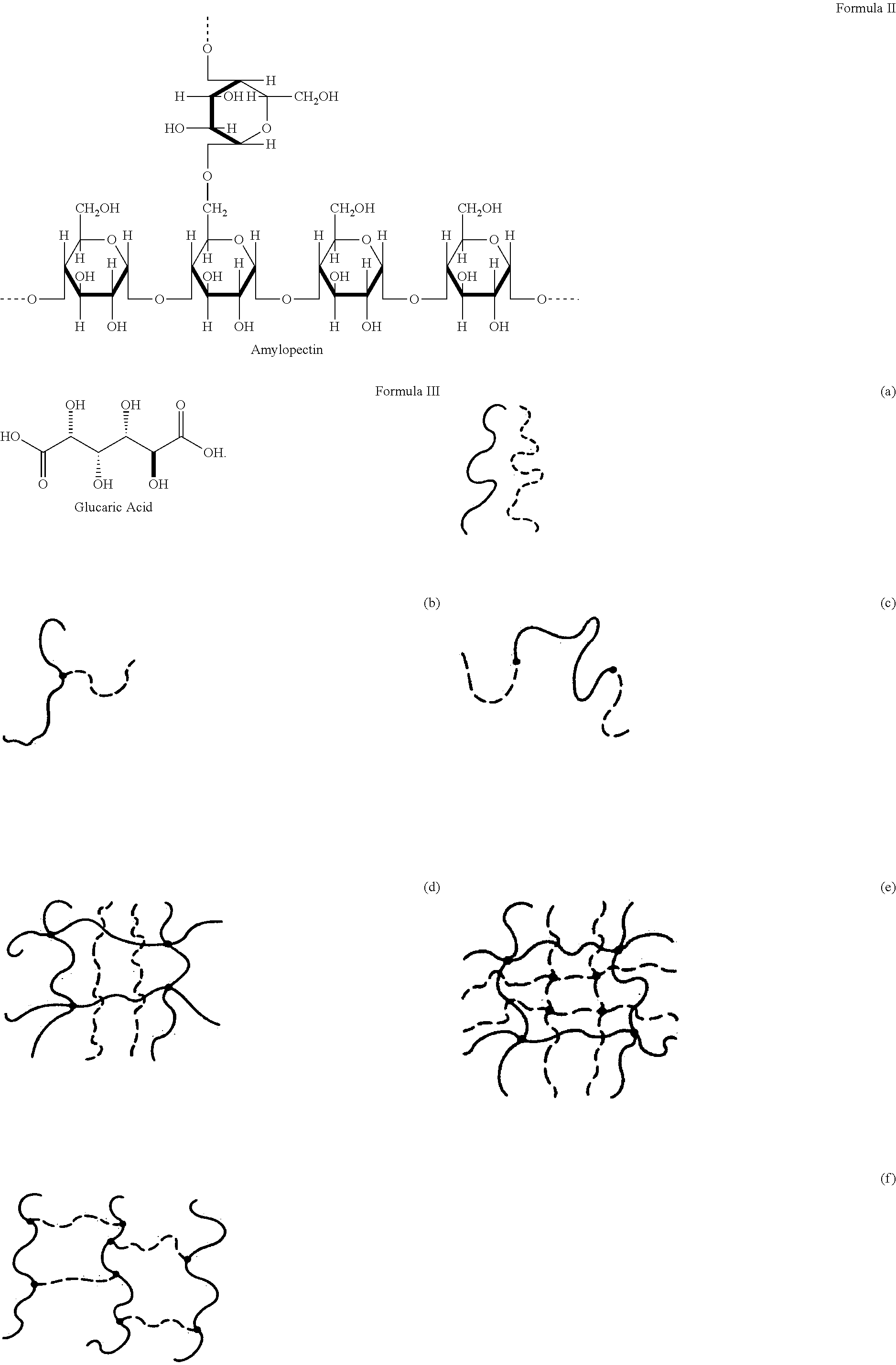

### Formation and Crosslinking of Network

In its native, or natural form, starch occurs as a granule made up of several alternating layers having crystalline and amorphous forms. The regions of long-range crystallinity appear to involve crystallization of the amylopectin component of starch, whereas the amylose component represents the amorphous phase of starch granules (Dumitriu 1996).

Amylose is a polysaccharide composed of unbranched chains of D-glucose units joined by alpha-1,4′-glycosidic linkages. The structural characteristics of amylose gels (2%-8% w/vol) at different scales of organization have been studied by electron microscopy, mild acid hydrolysis, differential scanning calorimetry, and size-exclusion chromatography. Amylose gels have been shown to exhibit a macro-porous structure, with a mesh size of 100-1000 nm, containing filaments 20+10 nm wide. These filaments result from the association of segments of amylose chains that are oriented obliquely to the filament axis. These amylose fragments are partially organized in a B-type crystalline array. Upon acid or enzymatic treatments, the amorphous segments of amylose chains are hydrolyzed (Dumtriu 1996).

In contrast to amylose, amylopectin is a branched polysaccharide. Like amylose, amylopectin is composed of chains of D-glucose units joined by alpha-1,4′-glycosidic linkages. Unlike amylose, amylopectin also contains alpha-1,8-glycosidic linkages, which create branches in the polysaccharide. Physical techniques such as x-ray diffraction (XRD), small angle neutron scattering (SANS), small angle x-ray scattering (SAXS), and electron microscopy (EM), have provided information on the nature of amylopectin crystallites. Such studies have shown a periodicity in native starch occurring at 10 nm, which is thought to be the repeat distance between the amorphous and crystalline regions (Dumitriu 1996). Amylopectin is a high molecular weight natural polymer with non-random branches. The branching of the polymer is extensive, and is responsible for the molecule's large hydrodynamic volume and ability to gel at concentrations of 3% and above in aqueous solution. The branching is known to be non-random; however, there is disagreement as to the precise architecture of the molecule because of the influence of steric hindrance and the placement of inner and outer chains. One widely accepted theoretical model of the molecular structure of amylopectin proposed by Durrani and Donald (1995) depicts a cascade-type branching structure, with the amylopectin branches arranging themselves in clusters of tiered branches.

In some embodiments of the present invention, the starch-derived polymeric network comprises or is derived from soluble starch. As discussed above, “soluble starch” refers to starch molecules that have been degraded. Soluble starch is derived from naturally occurring starch, for example, by acid hydrolysis with hydrochloric acid, resulting in a product having a molecular weight of about 300 g/mole. As mentioned, amylopectin and soluble of starch are both long chain molecules composed of glucose monomers; however they differ greatly in size. In contrast to soluble starch, the molecular weight of amylopectin can be as high as 10⁸ g/mole. Starch-based amygels comprising polymer networks made from soluble starch as a starting material are prepared in same way as those made from amylose and/or amylopectin.

A plurality of different three-dimensional structures can be formed when two polysaccharides are mixed together and gelled to form an Amygel in accordance with the instant invention. The chemical structures of various embodiments of the inventive amygels are dependent on the nature of the starting components, the rate and extent of de-mixing, and the method or mechanism of gelation.

The simplest type of structure is obtained when a first component forms a network, and a second component is merely contained within the first component. In more complex amygels in accordance with the invention, at least three types of gel structure can occur if both polysaccharides contribute to the network. In one type, the gel is an interpenetrating network in which both polysaccharides associate independently to form separate networks, but interlace with each other. In a second structural variation, a phase-separated network results if some degree of de-mixing occurs prior to gelation, and the two networks are spatially separated. In yet a third type of polysaccharide amygel structure, a coupled network is formed if the two networks chemically bond to each other during gelation.

One preferred embodiment of a naturally-derived polymer amygel suitable for drug delivery in accordance with the present invention is an amygel-based drug delivery system comprising starch, as described above. The Amygel comprises an interpenetrating network of physically entangled amylopectin and soluble starch polymer chains, chemically cross-linked by a cross-linker.

Based on study of the properties and characteristics of such amygels, as described in the Examples supra, it is believed that as the starch chains are heated, dispersed and stirred to prevent sedimentation during synthesis of the gels, the randomly coiled starch chains uncoil and become hydrolyzed, then recoil upon cooling, creating physical entanglements among neighboring chains. Also, as the dispersed starch chains are heated in solution, the crosslinker binds to the main chain backbone.

Gelation of Starch Amygels

“Gelation,” as the term is used generally herein, refers to the cross-linking of a plurality of polymer chains into progressively larger branched polymers, up to and including a single molecule spanning an entire system. “Starch gelation” as the term is used herein, refers to a process by which solubilization, hydration, and swelling of starch molecules occurs. For example, starch can be heated in a solution, during which it undergoes dispersion, randomly coiled chains uncoil and become hydrolyzed, and then recoil upon cooling, creating physical entanglements among neighboring chains. See further description, for example, in Whistler (1984).

Starch gelation can also occur by hydrogen bonding. Next to physical entanglement, hydrogen bonding is the second most important mechanism of starch gelation.

A hydrogen bond can form between two neighboring water molecules only if a hydrogen molecule lies close to the line connecting the oxygen atoms. This constraint prevents the starch molecules from packing as efficiently as they would if their interactions were solely due to dispersion forces and dipole-dipole interactions. During the gelation of starch in water, hydrogen bonding occurs around the perimeter of the starch helices, as well as the within the alpha helix. The alpha helix is capable of accommodating as many as 32 interstitial water molecules per turn of the alpha helix.

The phenomenon of “polymer immiscibility” is well known in the art of polymer chemistry and arises as a result of the generally unfavorable interactions between polymer species. Even a small positive free energy of interaction between different polymer species can result in limited miscibility due to the small entropy gain on mixing these high-molecular weight species. The miscibility of polymers in solution decreases with increasing polymer concentration, and is rare at high concentrations. If the segregation factor is strong, de-mixing is predicted when the polymer chains start to become entangled above the coil-overlap threshold. The phase behavior of ternary systems (i.e., polymer 1+polymer 2+solvent) can be strongly affected by polymer-solvent interactions. In general, immiscibility is increased when the affinity of one polymer for the solvent is significantly different from that of the other. Like synthetic polymers, biopolymers also exhibit immiscibility, with perhaps the best known example being the gelatin-gum Arabic-water system. Compared to synthetic polymer mixtures, there is relatively little information on the phase behavior of polysaccharide mixtures. In general, lower molecular weight species appear to have more affinity for the solvent.

As discussed, starch occurs naturally in seeds as a mixture of amylose and amylopectin molecules which are organized into a semi-crystalline granule. The behavior of aqueous solutions of starch is of interest because starch is processed by heating in the presence of water. Whereas amylose is essentially a (1-4) alpha-D-glucan, amylopectin is a (1-4) alpha-D-glucan with an average of one in every 20-25 residues branched at position 6. Because these polymers are chemically quite similar, it might be expected that immiscibility would only be observed at very high concentrations. However, it has been found that even moderately concentrated aqueous solutions of amylose and amylopectin exhibit immiscibility (Kalichevsky 1987).

According to Kalichevsky's theoretical study of mixtures of amylose and amylopectin of varying concentrations from pure amylose to pure amylopectin in a 10% total aqueous solution, the binodal phase diagram is not symmetric. Rather, it is shifted towards the amylose-rich phase. This is consistent with the behavior predicted for a mixture containing polymers of unequal molecular weight, the bimodal being displaced towards the polymer of lower molecular weight. As discussed by this author, the tie lines between phases at equilibrium with each other slope up to the amylose-rich phase, indicating that this phase has a higher affinity for the water.

The investigation of the phase behavior of amylose and amylopectin, using amylose of different molecular weights and structures as well as different temperatures (70 C-90 C), shows that these factors do not strongly affect the unfavorable interaction between these polysaccharides which gives rise to the phase separation. It is noted that the differences in molecular weight between the amylose samples are small, relative to the large differences between amylose and amylopectin. From the observation that phase separation only occurs at concentrations well above C*, (defined as minimum polymer concentration to induce phase separation), it is apparent that the segregation factor is not very strong. It has been observed that immiscibility becomes greater with increasing molecular weight, so the high molecular weights of these polysaccharides (especially the amylopectin), encourage phase separation.

The incompatibility of the linear and branched polymers of starch in aqueous solution has important implications. Incompatibility may also affect the types of interactions which can occur and should be considered as part of the gelatinization behavior of starch on heating in water (Kalichevsky 1987).

As shown in the Examples infra, the ratio of the polysaccharides (e.g., of amylose to amylopectin) in a starch-derived amygel can influence the properties of the polymeric network. Accordingly, the performance of the amygel can be altered by changing the ratio of the polysaccharides. For example, the susceptibility of the amygel to hydrolytic or enzymatic degradation can be altered by adjusting the amylose:amylopectin ratio in an exemplary amygel synthesized from these two starch components as starting materials.

Cross-Linking of Polysaccharides in Starch-Based Amygels

A factor affecting the performance of an amygel in accordance with the present invention is the degree of cross-linking that exists within the polymeric network. A “cross-link,” as the term is used herein, refers to a crosswise connecting part (as an atom or group) that connects roughly parallel chains in a complex chemical molecule (e.g., a polymer). In the majority of instances, a cross-link is a covalent structure, but the term is also used to describe sites of other chemical interactions (e.g., ionic interactions), portions of crystallites, and even physical entanglements.

The process of creating cross-linking in a starch-derived polymeric network is generally known in the art and typically involve the use of a “cross-linker,” also referred to as a “crosslinking agent” or a “cross-linker molecule.” “Chemical cross-linking” is defined as a linking mediated by the reaction of a linear or branched polymer with at least one cross-linking agent of relatively small molecular weight. As discussed, the function of the cross-linking agent is to link two larger molecular weight chains through its di- or multifunctional reactive chemical groups (Ratner 2004). The cross-linking agents can be added to the polymer solution after the polymer has been produced, the reactive species of the agents reacting with the polymer chains to form a chemically cross-linked system.

“Copolymerization cross-linking” refers to a reaction between a solution of one or more types of monomers including one multi-functional monomer that is present in relatively small quantities, where the polymerization of the polymer and the chemical cross-linking of the system occurs in one step. Another method involves combining monomer and linear polymeric chains that are cross-linked by means of an interlinking agent (Ratner 2004).

Cross-linking is a well established method for chemical modification of polymers. Varying degrees of cross-linking can be introduced into polysaccharides depending on the purpose, e.g., to generate larger molecular aggregates with enhanced viscosity profiles, or to enable preparation of insoluble products with a wide range of swelling characteristics (Dumitriu 1996) Some common cross-linking agents include bi- and tri-functional reagents, such as epichlorohydrin, bisepoxides, dihalogenated reagents, glutaraldehyde, acetaldehyde, formaldehyde, maleic and oxalic acid, dimethylurea, polyacrolein, diisocyanates, divinyl sulfate, ceric redox systems, and s-triazine.

There are several factors to consider when determining appropriate conditions for chemically cross-linking a starch-based amygel in accordance with the present invention. For example, the extent of the reaction and the number and character of side reactions must be considered. An important parameter that can be used for identification of the final cross-linked structure is the cross-linking ratio (CR), which is defined as the ratio of the moles of cross linking agent to the moles of polymer repeating units (Dumitriu 1996). For bi-functional crosslinking agents, the number average molecular weight between cross-links, M_{c}, may be determined by the relation M_{c}=Mᵣ/2CR where Mᵣ is the molecular weight of the repeating unit.

It is generally appreciated that the addition of a chemical cross-linker is important for improving both the integrity of an amygel and the predictability of its mechanical properties. Starch gels alone are inconsistent mechanically, due to the extent of phase separation, formation of aggregates, and induced crystallinity of the system. One important and recognized aspect of biopolymer gelation is the relationship between gel modulus and concentration. This relationship tests network theories which relate the molecular structure of the gel to the most apparent macroscopic aspect of the gel, i.e. its mechanical properties (Durrani and Donald 1995).

Various methods can be utilized in making an amygel that comprises a cross-linked starch-derived polymeric network. For example, in one method, a mixture including starch and DMSO is heated to at least partially solubilize the starch-polymer chains. During heating, the polymer chains of the starch components, which for example may include amylose and amylopectin, partially unravel and begin to entangle with neighboring chains resulting in the formation of the network. The heating also encourages the helical regions of the polymeric network to uncoil. This unraveling and uncoiling of the starch molecules allows the DMSO in the mixture to more readily permeate and solvate the network. Additionally, some of the glycosidic linkages in the network are hydrolyzed, further encouraging the unraveling/uncoiling and, hence, the solvation of the network. This synthesis process results in the formation of a homogeneous network dispersed in the DMSO solvent.

To produce a chemically crosslinked starch derived polymer network, a crosslinking agent can for example be added to the prepolymer solution, and using similar synthesis processes the functional groups of the crosslinker will chemically bind to the main chain backbones of the physically entangled starch molecules creating at least two types of crosslinks within the system. The first would be the physically entangled neighboring chains, or aggregates, that are the tie junctions of the starch network, and the second would be the tie junctions of the network formed by the chemical bonds between the starch chains and the crosslinking agent.

The temperature(s) attained at this stage of heating may vary, depending, inter alia, on several factors including the identity of the cross-linking agent; the concentration of the crosslinking agent in the mixture; and the desired degree of cross-linking in the polymeric network. Thereafter, the mixture is cooled, encouraging the polymer chains in the network to further entangle and coil. At least some of the DMSO solvent present in the network upon commencement of the cooling is retained therein, leading to the formation of the Amygel.

In some preferred embodiments of cross-linked Amygels in accordance with the invention, the linkage occurs by ester linkages. Ester linkages are preferred for this purpose because esters degrade hydrolytically, giving the gel its biodegradable properties. Typically, ester linkages are formed from the condensation reactions of alcohols and carboxyls. For example, in some preferred embodiments of the inventive Amygels, the cross-linker molecules utilize alcohol groups present in starch derivatives such as amylose and amylopectin.

The range of molecules that can be effectively utilized as cross-linkers in an Amygel of the present invention is not particularly limited. Any compound having at least two functional groups (carboxyl moieties) can be used as a chemical cross-linker to react, e.g., with the alcohol groups of the starch derivatives. Cross-linkers suitable for use in the present invention include, but are not limited to: dicarboxylic acids (e.g., oxalic acid, malonic acid, succinic acid, glutaric acid, adipic acid, pimelic acid, suberic acid, azelaic acid, sebacic acid, pthalic acid, isophthalic acid, and terephthalic acid; tricarboxylic acids (e.g., citric acid, isocitric acid, aconitic acid, and propane-1,2,3-tricarboxylic acid); and alpha hydroxyl acids, e.g., tartaric acid.

In some of preferred embodiments, the Amygel comprises a starch-derived polymeric network that is covalently cross-linked using D-glucaric acid lactone (e.g., D-glucaro-1,4-lactone) as a cross-linker. For example, the carboxyl group of D-glucaro-1,4-lactone that includes the C-6 carbon can react with a primary hydroxyl group on a first polymer chain of amylose or amylopectin via an esterification reaction, thereby resulting in a covalent bond between the two. Additionally, the pendant lactone can react with a primary hydroxyl group on a second polymer chain of amylose or amylopectin via a transesterification reaction, resulting in a covalent bond between these two, thereby completing the cross-link.

It is noteworthy that ester linkages are susceptible to hydrolysis, as are the glycosidic linkages in amylose, amylopectin, and other polysaccharides. Accordingly, a Amygel that comprises a starch-derived polymeric network that is cross-linked via an esterification process is “biodegradable,” meaning that the Amygel will erode or degrade in vivo, essentially yielding only biocompatible substances, including the cross-linker molecule.

It will be readily apparent to those of skill in the art of polymer production that this process can also be used to synthesize a starch-derived polymeric network that is cross-linked with any of the above-described cross-linkers.

Methods for testing the effects of cross-linkers on starch gel characteristics are known and have been described, for example, in studies of a product known as Cross-linked High Amylose Starch (CLHAS). CLHAS was introduced into the market several years ago as the controlled release device, Contramid® (Labopharm Inc, Laval, Quebec, Canada). Contramid® is a starchbased gel of made of amylose as the starch component, cross-linked with epichlorohydrin. Unlike the Amygels of the instant invention, this product, which can be administered orally or implanted subcutaneously, is designed to undergo gelation in vivo, i.e. only after it is placed in a biological environment. When placed in solution, the product swells to form an elastic gel.

As mentioned, the native starch granule is heterogeneous both chemically (comprising both amylose and amylopectin) and physically (having both crystalline and amorphous regions). The presence or absence of crystalline order is often a basic underlying property of starch. Depending on their origins, various types of native starches present specific morphologies, giving distinctive X-ray powder patterns termed types “A,” “B,” or “C” polymorphs. The sharpness of the X-ray diffraction pattern of starch granules depends on their water content, with the B-type being more sensitive to hydration than the A-type starch. The role of crystallinity in release control has been studied in a series of powders, tablets, and films of high amylose starch having varying degrees of cross-linking (Ispas-Szabo et al.).

Certain preferred methods of achieving cross-linking in Amygels are described in further detail in the Examples infra, and in Barker ED., “The Synthesis and Characterization of a Novel Polysaccharide Amygel for Biomedical Applications Including the Treatment of Malignant Tumors and the Prevention of Metastatic Disease,” Masters Thesis, University of Tennessee, first publicly available Jun. 26, 2008, herein incorporated by reference in its entirety.

In certain preferred embodiments of the invention, the cross-linker in the amygel is in itself a biologically active agent. As a non-limiting example, there is a significant body of evidence tending to establish that D-glucaro-1,4-lactone is a biologically active agent. It has been suggested, e.g., that D-glucaro-1,4-lactone possesses anti-cancer activity, among other beneficial biomedical effects (see, e.g., Walaszek, 1990). Certain preferred embodiments of amygels in accordance with the present invention comprise a starch-derived polymeric network that is cross-linked with D-glucaric acid (e.g., D-glucaro-1,4-lactone) and accordingly may have application for the treatment of cancer.

Amygels prepared in accordance with the present invention are suitable for parenteral administration to a warm-blooded organism, preferably a mammal, and most preferably a human. In one particularly preferred embodiment, the amygel is formulated as an injectable gel. Upon administration, (e.g., subcutaneously), the amygel can be used to increase the local concentration of a drug or therapeutic agent in the tissue. Other preferred embodiments can be administered parenterally by other delivery routes, and can facilitate the selective targeting of a location in the organism such that, at least initially, the concentration of the drug at that location can be increased substantially, relative to the overall concentration of the drug or therapeutic agent in the organism. Thus, as one example, an amygel comprising a starch-derived polymeric network that is loaded with an anti-cancer drug can be administered locally, e.g., by injection, to a location in proximity to a tumor requiring treatment.

In certain preferred embodiments of the present invention, the amygel comprises a starch-derived polymeric network that is cross-linked with a biologically active form of D-glucaric acid (e.g., D-glucaro-1,4-lactone). The cross-link in this instance can serve at least two purposes. In addition to causing a biological effect upon release from the amygel as described above, such a cross-link can serve to alter the degradation properties of the amygel. Accordingly, the degree of cross-linking affects the release kinetics of the biologically active form of D-glucaric acid. A suitable degree of cross-linking can, for example, increase the reliability of the performance of the amygel, insofar as release kinetics are concerned. It is worth noting that, in this instance, the degree of cross-linking acquires even greater significance because it is directly related to dosage.

Other factors affecting release kinetics from the amygel include: the ratio of amylose to amylopectin; and the density of the amygel. Suitable formulations can be determined by one skilled in the art and will vary depending, inter alia, on the route of administration; the desired biological effect (e.g., anti-cancer activity); and the desired release kinetics (e.g., sustained release and other types of controlled release). The particular formulations also may vary, depending on whether the desired biological effect is therapeutic, prophylactic, or diagnostic.

By controlling the degree of cross-linking during synthesis, it is possible to achieve distinct degradation properties and drug release profiles. During synthesis, specific concentrations of starch are added to organic solutions of the chemical cross-linker. The gel network is then formed as the pre-polymer solution is heated. Upon heating, the helices of the polymer chains open up and entangle with neighboring chains, forming aggregates. The starch components are immiscible in solution due to differences in solubility parameters, as governed by the various molecular weights of the chains present. This phenomenon induces phase separation within the system. Accordingly, like chains physically entangle with each other during the process and an interpenetrating network of the high and low molecular weight chains is formed. At the same time, the carboxyl groups of the chemical cross-linker react with the alcohol groups of the starch via condensation, creating ester linkages that covalently tie the network together.

The degree of cross-linking within the system determines the pore size of the network, a factor that is important with respect to release of therapeutic agent from within the system. By increasing or decreasing both the physical and/or chemical crosslink density of the polymer, the pore size of the network can be altered in a predictable and reproducible way.

Starch-Based Amygels Comprising Biologically Active Agents

In some embodiments of the present invention, the dispersed phase (colloid) of the amygel includes at least three different chemical substances: a starch-derived polymeric network; a cross-linking agent; and a biologically active agent.

The starch-derived polymeric network is cross-linked to facilitate the controlled release in vivo of the biologically active agent. In at least some of these embodiments, the starch derived polymeric network is cross-linked also to facilitate the retention in vitro of a biologically active agent. The biologically active agent can complex with the starch-derived amygel by one of several mechanisms. The biologically active agent can be incorporated into the matrix of the gel network. Alternatively, the biologically active agent can complex within the molecular structure of the individual polymer chains comprising the network. The biologically active agent can also react with the main chain backbone of the polymers comprising the network. The biologically active agent can be loaded into the amygel either during the synthesis of the network, or after the network has formed, depending upon the type of agent being loaded. For instance, a protein-like biological agent would not be able to withstand the heat required to form the amygel network, so it can be added after the gel has been formed.

To load the gel after synthesis, a simple diffusion method can be used to dissolve the biologically active agent in DMSO and add the gel to the solution. The active agent then diffuses into the gel and becomes complexed within the gel matrix.

The cross-link can change the pore size of the matrix, thereby impeding diffusion of the biologically active agent out of the gel matrix. The cross-link can in some instances change the mechanical properties of the starch-derived polymeric network, thereby enhancing the ability of the network to retain biologically active agents. Such changes can affect the helical regions of the starch-derived polymeric network, allowing these regions to more readily accept and retain a biologically active agent (e.g., an antibiotic, or a drug to be delivered to a particular site in the body, for example). It is worth noting that in embodiments in which the biologically active agent is complexed with the helical or other regions of the starch-derived polymeric network, diffusion can be a significant factor in effecting the release of the biologically active agent from the Amygel. Thus, one skilled in the art will consider the process of diffusion as well as the degradability of the Amygel when formulating and designing such an amygel.

Biocompatibility of amygels in accordance with the present invention can further include other natural polymers such as lipids, proteoglycans, or polysaccharides. One of the most advantageous features of natural polymers in this regard is the ability of these polymers to be degraded by naturally-occurring enzymes and metabolized by normal physiological mechanisms (Dumitriu S, Polysaccharides in Medical Applications, Marcel Dekker Inc., New 30 York, N.Y., 1996).

One preferred embodiment of a naturally-derived polymer amygel suitable for drug delivery in accordance with the present invention is an amygel-based drug delivery system comprising starch as described above. The amygel comprises an interpenetrating network of physically entangled amylopectin and soluble starch polymer chains, chemically cross-linked by a cross-linker.

### EXAMPLES

The invention is further described by the following non-limiting Examples.

### Example 1

### Synthesis of Starch-Based Amygels

A starch-based amygel for biomedical applications in accordance with the present invention comprises a biodegradable gel network that is cross-linked by ester linkages which can be degraded hydrolytically. This Example describes the synthesis of exemplary amygels in accordance with the present invention, in which alcohol groups on starch derivatives (amylose, amylopectin, or soluble starch) are reacted to form two ester linkages, one on each end of glucaric acid (GA), which is used as a chemical cross-linker.

### Example 2

### Characteristics of Starch-Based Amygels

This example describes the characteristics of a plurality of embodiments of starch-based amygels made in accordance with the present invention. In various embodiments, the starchbased amygels comprise: amylopectin as the only starch component; amylose as the only starch component; soluble starch as the only starch component; a “starch composite” including any combination of amylopectin, amylose, or soluble starch; and amylopectin/amylose/soluble starch composites that are cross-linked with varying concentrations of an exemplary cross-linker (glucaric acid, GA).

### LITERATURE CITED

It is believed that a review of the following references will enhance understanding of the present invention.
Dumitriu, S. Polysaccharides in Medicinal Applications. Marcel Dekker, Inc. New York: 30 1996.
Flory, P. J. Statistical Mechanics of Chain Molecules. Oxford University Press, New York: 1969. Whistler, BeMiller, and Paschall. Starch Chemistry and Technology, Second Edition. Academic Press, Inc. London: 1984.
Kalichevsky, M. T. and Ring, S. G., “Incompatibility of Amylose and Amylopectin in Aqueous Solution,” Carbohydrate Research. Vol 162, pgs 323-328, 1987.
Araujo, M. A., et al. “Enzymatic Degradation of Starch-based Thermoplastic Compounds used in Prostheses: Identification of the Degradation Products in Solution,” Biomaterials. Vol. 25, pgs 2687-2693, 2004.
Ratner, Hoffman, Schoen, Lemons. Biomaterials Science: An Introduction to Materials in Medicine 2nd Edition. Elsevier Academic Press. New York: 2004.
Anseth, K., Bowman, C., Peppas, L. “Mechanical Properties of Amygels and their Experimental Determination.” Biomaterials vol 17. 1996. pp. 1647-1657.
Durrani and Donald. “Physical Characterization of Amylopectin Gels.” Polymer Gels and Networks vol 3. 1995. pp 1-27.
Morawetz, Herbert. Macromolecules in Solution. John Wiley and Sons. New York: 1965.
Morrison, R. T and Boyd, R. N. Organic Chemistry, Fourth Edition. Allyn and Bacon, Inc. Boston: 1983.
Walaszek, Z. “Potential Use of D-Glucaric Acid Derivatives in Cancer Prevention,” Cancer Letters, Vol. 54, No 1-2, pgs. 1-8, 1990.
Callister, William D. Materials Science and Engineering: an Introduction. John Wiley and Sons. New York: 2003.
Morrison, Faith. Understanding Rheology. Oxford University Press. New York: 2001. Durrani and Donald. “Shape, Molecular Weight Distribution and Viscosity of Amylopectin in Dilute Solution.” Carbohydrate Polymers. v 41 2002. pp 207-217.
Horton, D. and Walaszek, Z. “Conformation of the D-glucarolatones and D-glucaric Acid in Solution.” Carbohydrate Research, 105 (1982) 95-109.
Ispas-Szabo, P., Ravenelle, F., Hassan, I., Preda, M., Mateescu, M., “Structure-Properties Relationship in Crosslinked High Amylose Starch for Use in Controlled Drug Release.” Carbohydrate Research. 323 (2000) 163-175.

What is claimed is:

1. An Amygel for the controlled delivery of a biologically active agent comprising:
a dispersed phase including a polymeric network comprising a hydrolysable polymeric substance derived from starch, and a cross-linker; and
a dispersion medium comprising an organic solvent.

2. The Amygel of claim 1, wherein said hydrolyzable polymeric substance derived from starch is amylose, amylopectin, or a combination thereof.

3. The Amygel of claim 1, wherein said hydrolyzable polymeric substance derived from starch is soluble starch.

4. The Amygel of claim 1, wherein the cross-linker is covalently bound to the polymeric substance derived from starch by means of an ester linkage.

5. The Amygel of claim 1, wherein the cross-linker is a molecule having at least two carboxyl moieties, selected from a dicaroxylic acid, a tricarboxylic acid, and an alpha hydroxyl acid.

6. The Amygel of 4, wherein the cross-linker is selected from the group consisting of D-glucaric acid, a derivative of D-glucaric acid, or an analogue of D-glucaric acid.

7. An Amygel for the controlled delivery of a biologically active agent comprising:
a dispersed phase including a hydrolyzable starch-derived polymeric network comprising a combination of amylose and amylopectin and a cross-linker; and
a dispersion medium comprising an organic solvent.

8. The Amygel of claim 7, wherein the cross-linker is covalently bound to the starch-derived polymeric network by means of an ester linkage.

9. The Amygel of claim 7, wherein the cross-linker is a molecule having at least two carboxyl moieties, selected from a dicaroxylic acid, a tricarboxylic acid, and an alpha hydroxyl acid.

10. An Amygel for the controlled delivery of a biologically active agent comprising:
a dispersed phase including a polymeric network comprising a hydrolyzable polymeric substance derived from starch, and
a cross-linker; and a biologically active agent; and
a dispersion medium comprising an organic solvent.

11. The hyrogel of claim 10, wherein the biologically active agent is covalently bound to the polymeric network.

12. The hyrogel of claim 10, wherein the biologically active agent is noncovalently associated with the polymeric network.

13. The Amygel of claim 10, in wherein the biologically active agent is a small molecule.

14. The Amygel of claim 10, wherein the biologically active agent is a macromolecule selected from a protein, a polysaccharide and a nucleic acid.

15. The Amygel of claim 10, wherein the polymeric network is biodegradable.

16. The Amygel of the claim 10, wherein upon administration of said Amygel to a host animal, the products released from said Amygel during biodegradation are only naturally-occurring molecules that are nontoxic and nonimmunogenic to said host animal.

17. The Amygel of claim 10, formulated as an injectable.

18. The Amygel of claim 10, wherein the cross-linker is in itself a biologically active agent.

19. The Amygel of 18, wherein the cross-linker is a selected from the group consisting of D-glucaric acid, a derivative of D-glucaric acid, or an analogue of D-glucaric acid.

20. The Amygel of claim 10, wherein the Amygel has a viscous, jelly-like texture.
